(19) United States
(12) Patent Application Publication
(10) Pub. No.: US 2017/0182069 A1
(43) Pub. Date: Jun. 29, 2017

(54) TOPICALLY APPLICABLE CHEMICAL PEEL COMPOSITION

(71) Applicant: L'OREAL, Paris (FR)

(72) Inventors: Gloria LU, Rahway, NJ (US); Patricia BRIEVA, Manalapan, NJ (US); Donna McCANN, Oxford, NJ (US)

(21) Appl. No.: 14/982,220

(22) Filed: Dec. 29, 2015

(51) Int. Cl. A61K 31/60; A61K 31/05; A61Q 19/08; A61K 8/34; A61Q 19/02; A61K 31/19; A61K 8/365

(52) U.S. Cl. CPC A61K 31/60; A61K 31/19; A61K 31/05; A61K 8/365; A61K 8/347; A61Q 19/02; A61Q 19/08

(57) ABSTRACT

A topically applicable chemical peel composition for home use having from about 4% to about 13%, by weight, of alpha hydroxy acid, from about 0.1% to about 2.0%, by weight, of salicylic acid, from about 0.1% to about 2.0%, by weight, of phenylethyl resorcinol and balance essentially dermatologically acceptable liquid solvent. In addition, a chemical skin peel regime or regimen including topical application onto the skin is also disclosed.

### FIELD OF THE INVENTION

The present invention relates to methods of peeling skin using a certain resorcinol derivative, to chemical skin peel compositions comprising this certain resorcinol derivative in a carrier, preferably a dermatologically acceptable carrier, to methods of making these compositions, and methods of applying this certain derivative and/or composition to skin to be peeled.

### BACKGROUND OF THE INVENTION

Known skin peeling procedures include mechanical removal, such as, dermabrasion or CO₂ laser, and chemical-induced skin removal. Chemical-induced skin peeling techniques or chemical peels are widely utilized and have a variety of types that provide varying degrees of skin removal.

Chemical peels are a non-invasive dermatological treatment techniques to improve and treat skin conditions including: photodamaged skin, hyperpigmentation, acne vulgaris, rosacea, premalignant skin cancer, wrinkles and fine lines, superficial scars and the like. Chemical peel works by applying a solution of acids topically; the acids then penetrate into the skin, inducing and accelerating skin's natural regeneration process by sloughing off the dead top layers of skin. The strength and efficacy of the peel is dictated by the formulation, acid type and concentration, and the depth of penetration. Common chemical peels actives include: glycolic acid, lactic acid, salicylic acid, trichloroacetic acid (TCA). The strength and efficacy of the peel is dictated by the formulation, acid type and concentration, and the depth of penetration.

Peels with high levels of acid (greater than 30%) and extreme low pH (less than 3) are considered professional use, and could only be distributed by licensed dermatologists and/or aestheticians. However, recently, use of chemical peels have begun to penetrate into the home-use market. Typically, home use peels have a total alpha hydroxy acid (glycolic and lactic acid) concentration of less than 10%, with a final formula pH greater than 3.5. These home use peels tout clinical efficacy with mild formulations safe enough for self-application at home. The challenge with home peels lies in delivering clinical-like efficacy with the lower acid level and elevated formulation pH.

Known professional peel compositions, such as Jessner peels (Salicylic+Lactic+Resorcinol) are popular and efficacious in professional settings. However, Jessner combination and Jessner-like peels have not penetrated the home peel market.

A resorcinol derivative, phenylethyl resorcinol, is known as a tyrosinase inhibitor, which has been incorporated in non-chemical peel serums at 0.3 to 0.5% to lighten and promote even skin tone. Resorcinol and phenylethyl resorcinol have significantly different structures and properties and phenylethyl resorcinol has not previously been utilized in chemical peels or at concentrations utilized in chemical peels. In addition, phenylethyl resorcinol has not been shown to have adverse effects in basic toxicological tests, including acute oral toxicity, mutagenicity, skin irritation, skin sensitization, and phototoxicity.

TABLE 1

| Molecular Structure | 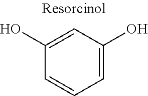 | 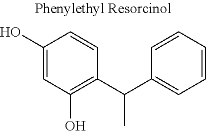 [SMILES: CC(c1ccccc1)c1ccc(O)cc1O] |
|---|---|---|
| MW | 110.11 | 214.27 |
| pKA | 9.32-9.81 | 9.77-10.77 |
| Log P | 2.47 | 2.11 |

Therefore, a need exists for a chemical skin peeling agent and composition and related topically applied technique that provides exceptional results, preferably without or with less adverse side effects or drawbacks found with conventional chemical peels, agents and compositions. In addition, a need exists for an intense, home-use level chemical peel that provides results better than traditional peels and that can visibly, significantly improve skin condition comprehensively after a series of a few applications.

### BRIEF SUMMARY OF THE INVENTION

One embodiment according to the present disclosure includes a topically applicable chemical peel composition from about 4% to about 13%, by weight, of alpha hydroxy acid, from about 0.1% to about 2.0%, by weight, of salicylic acid, from about 0.1% to about 2%, by weight of phenylethyl resorcinol and balance essentially dermatologically acceptable liquid solvent.

The present disclosure is also directed to a method for cosmetic treatment of keratinous tissues by applying the above-disclosed composition onto a surface of the keratinous tissue.

Other features and advantages of the present invention will be apparent from the following more detailed description of the preferred embodiment which illustrates, by way of example, the principles of the invention.

### DETAILED DESCRIPTION OF THE INVENTION

Provided are an exemplary chemical peel composition and methods for utilizing chemical peel composition. Embodiments of the present disclosure, in comparison to methods and products not utilizing one or more features disclosed herein, improve tolerability preferably without or with less adverse side effects or drawbacks found with conventional chemical peels, agents and compositions. The chemical peel composition, according to the present disclosure, provides reduced fine lines and wrinkles, reduced uneven pigmentation, and improved overall skin texture. In addition, the chemical peel provides better efficacy than existing home peel technologies.

Unless specifically defined, all technical and scientific terms used herein have the same meaning as commonly understood by a skilled artisan in biochemistry, chemistry, cosmetology, dermatology and materials science.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

All numbers expressing quantities of ingredients and/or reaction conditions are to be understood as being modified in all instances by the term “about”, unless otherwise indicated.

“Keratinous tissue,” as used herein, includes, but is not limited to, skin, hair, and nails.

In the present application, the term “ambient temperature” means a temperature of about 25° C.

The present invention includes a chemical skin peel composition comprising alpha hydroxy acid, salicylic acid, phenylethyl resorcinol and a carrier, preferably a dermatologically acceptable carrier. Preferably the composition is one designed to be, and capable of being, rinsed off after application.

Alpha Hydroxy Acid

The chemical peel composition, according to the present disclosure, includes alpha hydroxy acid. Suitable alpha hydroxy acids include lactic acid, glycolic acid, tartaric acid, mandelic acid, citric acid, ester derivatives thereof and combinations thereof. Exemplary ester derivatives include ester compounds of lactic acid, such as methyl lactate, ethyl lactate, butyl lactate and, similarly, ester compounds of glycolic acid, tartaric acid, mandelic acid, citric acid. One particularly suitable alpha hydroxy acid is lactic acid. Lactic acid, or 2-hydroxypropanoic acid, is provided to the chemical peel composition to provide enhanced exfoliation of the skin. In addition, lactic acid also boosts production of glycosaminoglycan (GAG) in the skin, improving the barrier function and moisturization of skin.

The chemical peel composition, according to the present disclosure, includes a concentration of alpha hydroxy acid, said composition being characterized in that it preferably has a concentration of alpha hydroxy acid of from about 4% to about 13%, or alternatively about 8% to about 12% or alternatively about 9% to about 11%, or alternatively about 10%, all percents being based on total weight of composition.

Salicylic Acid

The chemical peel composition, according to the present disclosure, includes salicylic acid. Salicylic acid, or 2-hydroxybenzoic acid, is provided to the chemical peel composition to provide enhanced penetration of the composition into the skin. Salicylic acid penetrates deeper into the skin than alpha hydroxy acids (AHAs), such as lactic acid. Salicylic acids is an effective keratolytic and comedolytic agent, inducing desquamation and can be used to effectively treat excessive oil, acne, post-inflammatory hyperpigmentation, and photodamage.

The chemical peel composition, according to the present disclosure, includes a concentration of salicylic acid, said composition being characterized in that it preferably has a concentration of salicylic acid of from about 0.1% to about 2.0%, or alternatively about 0.2% to about 1.5%, or alternatively about 0.3% to about 1.0%, or alternatively about 0.35% to about 0.75%, or alternatively about 0.4% to about 0.5%, or alternatively about 0.45%, all percents being based on total weight of composition.

Phenylethyl Resorcinol

The chemical peel composition, according to the present disclosure, includes phenylethyl resorcinol. Phenylethyl resorcinol has the following chemical formula:

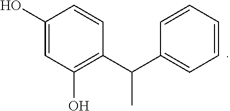 [SMILES: CC(c1ccccc1)c1ccc(O)cc1O]

Phenylethyl resorcinol functions a tyrosinase inhibitor. The phenylethyl resorcinol, when utilized in the composition according to the present disclosure effectively whiten skin and reduce skin tone unevenness.

Phenylethyl resorcinol has not shown adverse effects in basic toxicological tests, including acute oral toxicity, mutagenicity, skin irritation, skin sensitization, and phototoxicity.

The chemical peel composition, according to the present disclosure, includes a concentration of phenylethyl resorcinol, said composition being characterized in that it preferably has a concentration of phenylethyl resorcinol of from about 0.1% to about 2.0%, or alternatively about 0.2% to about 1.5%, or alternatively about 0.4% to about 1.0%, or alternatively about 0.6% to about 0.9%, or alternatively about 0.7% to about 0.8%, or alternatively about 0.75%, all percents being based on total weight of composition

Dermatologically Acceptable Liquid Solvent

A group of preferred carriers used for the chemical skin peel compositions of the invention comprising phenylethyl resorcinol are dermatologically acceptable liquid solvents in which the lactic acid, salicylic acid and phenylethyl resorcinol are soluble at high concentrations. The term “dermatologically acceptable liquid solvents” is intended to mean those solvents which can safely be used on the skin in the topical treatment method of this invention, i.e., solvents which do not provoke a severe reaction and which are not toxic when contacted with the skin for relatively short periods of time. Preferred solvents are organic solvents that are relatively volatile, to facilitate evaporation of the solvent after application of a coating of the salicylic acid derivative-containing solution onto the skin. Examples of preferred solvents include ethanol and isopropanol. Other useful solvents include methanol, acetone and ether (diethyl ether). In addition, in other embodiments, water may be utilized as solvent. In other embodiments, mixtures of one or more of these solvents or other solvents are included.

Ethanol is a particularly suitable solvent. The ethanol may be aqueous ethanol, preferably containing about 2.00 to 95.76 wt % ethanol or about 70.0 to 90.0 wt % ethanol or about 5.0 to 20.0 wt %. The ethanol employed as the solvent is preferably a grade of ethanol suitable for use in dermatological formulations. As indicated above for ethanol, carriers and solvents may contain contain water, which is preferably miscible with the solvent. The water may be present in an amount from 0 to about 87 wt %.

It is important to note that the above-mentioned dermatologically acceptable liquid solvents, whether preferred or not, may be utilized alone or in combination with one another. In addition, other useful carriers herein include the various dermatological and cosmetic carriers such as gels, emulsions, creams, waxes, compacts, etc.

Optional Additives

The compositions of the invention may of course comprise other components, such as preservatives, stabilizers, antioxidants, thickening agents, surfactants, pigments, colorants, fragrances and other adjuvants. Such components are preferably dermatologically acceptable. Preferably, the additional components do not interfere with the efficacy or impose any negative influence upon the efficacy of the chemical peeling agent. Such additives may further include, for example, an aromatic, a surfactant, a preservative, an anti-oxidant, a moisturizing agent, and so on. In addition, vitamin A acid, an alkyl acrylatemethacrylate copolymer, etc. may be added. Of course the additional components may be present individually or in combination, and their concentrations are not limited and may be, for example, from about 0.01 to about 5 wt %, based on the weight of the chemical skin peel composition. In one embodiment the amounts of such additional components are minimized so as not to cause a significant reduction in the maximum, i.e., saturation, concentration of salicylic acid derivative in the solvent.

The present invention also provides chemical skin peel compositions that comprise phenylethyl resorcinol as described above formulated so as to be acceptable to the consumer and, preferably, stable and/or clear.

The present invention also provides methods of making the above-mentioned chemical skin peel compositions by mixing the above described composition with at least one carrier such as a dermatologically acceptable carrier, where mixing includes all orders of addition. In one embodiment, the salicylic acid is dissolved in an alcoholic solvent at room temperature. Once salicylic acid is completely dissolved, the phenylethyl resorcinol is added to the solution and mixed to dissolve at room temperature. After the phenylethyl resorcinol is added, the lactic acid is added and the composition is mixed.

The composition, according to the present invention, may be applied in any manner, for example, by pasting, spraying, wiping, dispensing, etc. (hereinafter “applying”) the invention salicylic acid derivative(s) themselves or the invention chemical skin peel composition on the skin to be peeled. This can typically be accomplished with, for example, a spray bottle, an absorbent cotton swab wetted with the concentrated solution, with a solution-wetted sable brush or by gentle wiping with a solution-wetted absorbent fibrous material, such as a gauze square or nonwoven pad, but other solution application techniques that coat the skin with the solution, preferably in a uniform manner, are also feasible. The application serves as a peel, the degree of which depends upon the amount or concentration of acid compound and time of application, all of which are within the skill of the ordinary artisan in view of this disclosure. The compound(s)/composition of the invention may in addition be applied not only to the skin to be peeled but also to surrounding areas.

The compound(s)/composition of the invention is preferably applied to the skin to be peeled at a temperature of about 15° C. to about 30° C., about 20° C. to about 25° C. being preferred. Depending on carrier/solvent volatility characteristics, a temperature outside of these ranges, e.g., use of lower temperatures for highly volatile materials, may be preferred.

The composition is suitable for home-use or home application. As such, the composition according to the present invention is preferably applied by a non-professional or is self-applied in a non-clinical environment.

The applied compound or composition is normally allowed to air dry over a relatively short period of time, preferably being less than 15 minutes and, with the preferred ethanol solvent, typically being in the range of about 3 to 10 minutes. Drying may be promoted by directing a gentle stream of air, preferably warm air, onto the treated area or by other analogous procedures. A single uniform application of the composition to the skin to be treated and/or its surroundings is generally sufficient. Additional or multiple applications either before or immediately after the applied solution has dried are normally unnecessary but may be useful in some situations, e.g., in treating skin on other parts of the body other than the face or in treating skin severely in need of peeling.

Once the applied solution has been on the skin of, e.g., the face, for sufficient time, the compound(s)/composition can be removed from the skin, for example, after the composition has dried on the skin, or it may be allowed to remain on the skin for further time, depending on the results desired. When treatment is finished, the skin may thereafter be preferably wiped or washed, rinsed, etc., with water, etc., to remove any residue or traces of the applied salicylic acid derivative, composition or solution, including any deposits of salicylic acid derivative that may remain after drying.

Preferably, the treated skin is washed or wiped with water, e.g., with a water-moistened or water-wet swab, gauze square, or the like. Other solutions, such as an aqueous solution of mild detergent, aqueous alcohol solutions or isopropanol or ethanol, and the like, may also be used for this purpose. Additional applications of the concentrated salicylic acid derivative or composition immediately after the wiping/washing step, followed by drying and repeated wiping/washing, are generally unnecessary, as noted above, but may be desirable in some circumstances.

Subsequent to the peeling process, a bland or mild moisturizer may be applied, as desired, to the treated skin to reduce the visibility of scaling, peeling skin and to reduce skin dryness. The skin treated in the method of this invention may be treated further, with conventional skin treatment therapies. In one embodiment, the skin may be treated with a photoprotective product with sufficient sun protection factor (SPF) and or broad spectrum protection to reduce or eliminate photodamage to sensitized skin. In one embodiment, the skin may be treated with soothing agents, such creams, lotions and masques.

The compounds and compositions of the invention provide an advantage in that the treatment time, i.e., the period during which the treated skin is exposed to the phenylethyl resorcinol, is normally self-limiting and is not dependent on the intervention of the applicator for determining length of treatment time or determining when the treatment period should terminate. For relatively volatile solvents, such as ethanol, the evaporation of the solvent from the coating of chemical peel composition is effective for controlling the treatment time, ensuring not only constancy in treatment time, but also avoiding the need for applicator intervention to avoid excessively long exposure to the chemical peel composition comprising phenylethyl resorcinol.

The treatment time, according to this invention, is preferably measured as the time of exposure of the treated skin to the compound/composition, with treatment time ending for compositions once the carrier (e.g., volatile solvent) has evaporated from the applied solution or once the still-wet coating of applied composition is wiped or otherwise removed from the skin.

In one embodiment according to the present invention, the composition is applied using a two-step. The process includes applying the composition to the skin in a first step. Thereafter, a neutralizer, such as a sodium bicarbonate solution, having additional actives may be applied. These actives may include any compounds that provide a desirable cosmetic effect to the skin upon application. For example, actives may include, but are not limited to ascorbic acid, ascorbyl palmitate, retinol, and resveratrol.

In another embodiment according to the present invention, prior to application of the composition, the skin may be scrubbed to exfoliate the skin. Thereafter, the composition is applied to the skin and the chemical peel is provided.

In order to further illustrate the present invention and the advantages thereof, the following specific examples are given, it being understood that same are intended only as illustrative and in nowise limitative.

In said examples to follow, all parts and percentages are given by weight unless otherwise indicated.

### EXAMPLES

TABLE 1

| INCI (wt %) | Example 1 | Example 2 |
|---|---|---|
| Phenylethyl Resorcinol | 0.75 | 0.75 |
| Lactic Acid | 10.00 | 10.00 |
| SALICYLIC ACID | 0.45 | 0.45 |
| ALCOHOL DENAT. | 80.00 | 12.69 |
| WATER | q.s. | q.s. |
| TOTAL | 100.00 | 100.00 |

The Examples were prepared by dissolving salicylic acid in alcohol at room temperature. Once salicylic acid was completely dissolved, phenylethyl resorcinol was added to the solution, mixed and dissolved at room temperature. Lactic acid was added and mixed well.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A topically applicable chemical peel composition comprising:
from about 8% to about 12%, by weight, of alpha hydroxy acid;
from about 0.1% to about 2.0%, by weight, of salicylic acid;
from about 0.1% to about 2.0%, by weight, of phenylethyl resorcinol;
balance essentially dermatologically acceptable liquid solvent.

2. The chemical peel composition of claim 1, wherein the dermatologically acceptable liquid solvent includes ethanol.

3. The chemical peel composition of claim 1, wherein the composition includes from about 0.2% to about 1.5%, by weight, of phenylethyl resorcinol.

4. The chemical peel composition of claim 1, wherein the composition includes from about 0.6% to about 0.9%, by weight, of phenylethyl resorcinol.

5. The chemical peel composition of claim 1, wherein the composition includes about 0.75%, by weight, of phenylethyl resorcinol.

6. The chemical peel composition of claim 1, wherein the alpha hydroxy acid is selected from the group consisting of lactic acid, glycolic acid, tartaric acid, mandelic acid, citric acid, ester derivatives thereof and combinations thereof.

7. The chemical peel composition of claim 1, wherein the alpha hydroxy acid is lactic acid.

8. (canceled)

9. The chemical peel composition of claim 1, wherein the composition includes from about 9% to about 11%, by weight, of alpha hydroxy acid.

10. The chemical peel composition of claim 1, wherein the composition includes about 10.0%, by weight, of alpha hydroxy acid.

11. The chemical peel composition of claim 1, wherein the composition includes from about 0.3% to about 1.0%, by weight, of salicylic acid.

12. The chemical peel composition of claim 1, wherein the composition includes from about 0.4% to about 0.5%, by weight, of salicylic acid.

13. The chemical peel composition of claim 1, wherein the composition includes about 0.45%, by weight, of salicylic acid.

14. The chemical peel composition of claim 1, further comprising an additive selected from the group consisting of preservatives, stabilizers, antioxidants, thickening agents, surfactants, pigments, colorants, fragrances and combinations thereof.

15. A chemical skin peel regime or regimen including topical application onto the skin of the chemical peel composition of claim 1.

16. The chemical skin peel regime or regimen of claim 15 comprising treating photodamaged skin, hyperpigmentation, acne vulgaris, rosacea, wrinkles, fine lines or superficial scars.

17. A topically applicable chemical peel composition comprising:
from about 8% to about 12%, by weight, of alpha hydroxy acid;
from about 0.4% to about 1.0%, by weight, of salicylic acid;
from about 0.1% to about 2.0%, by weight, of phenylethyl resorcinol;
balance essentially dermatologically acceptable liquid solvent.

18. The chemical peel composition of claim 17, wherein the alpha hydroxy acid is selected from the group consisting of lactic acid, glycolic acid, tartaric acid, mandelic acid, citric acid, ester derivatives thereof and combinations thereof, and further comprising an additive selected from the group consisting of preservatives, stabilizers, antioxidants, thickening agents, surfactants, pigments, colorants, fragrances and combinations thereof.

19. A topically applicable chemical peel composition comprising:
about 10%, by weight, of alpha hydroxy acid;
about 0.45%, by weight, of salicylic acid;
about 00.75%, by weight, of phenylethyl resorcinol;
an additive selected from the group consisting of preservatives, stabilizers, antioxidants, thickening agents, surfactants, pigments, colorants, fragrances and combinations thereof, balance essentially dermatologically acceptable liquid solvent.

20. The chemical peel composition of claim 19, wherein the alpha hydroxy acid is selected from the group consisting of lactic acid, glycolic acid, tartaric acid, mandelic acid, citric acid, ester derivatives thereof and combinations thereof, and wherein the additive includes a solvent selected from denatured alcohol.

21. The chemical peel composition of claim 20, wherein the alpha hydroxy acid is lactic acid and the alcohol is ethanol.
